(19) United States
(12) Patent Application Publication
(10) Pub. No.: US 2012/0029394 A1
(43) Pub. Date: Feb. 2, 2012

(54) Ultrasound Assisted Laser Skin and Tissue Treatment

(75) Inventor: Eilaz BABAEV, Minnetonka, MN (US)

(73) Assignee: BACOUSTICS, LLC, Minnetonka, MN (US)

(21) Appl. No.: 13/021,962

(22) Filed: Feb. 7, 2011

Related U.S. Application Data
(60) Provisional application No. 61/301,683, filed on Feb. 5, 2010.

(51) Int. Cl. A61N 5/067; A61B 18/02; A61N 7/00

(52) U.S. Cl. 601/2

(57) ABSTRACT

The present invention relates to an ultrasound assisted medical laser for the therapeutic and cosmetic treatment of a patient's skin and underlying adipose tissue and more particularly, to apparatuses and methods using combinations of ultrasound, lasers and cryogenic energy to rejuvenate and condition skin and related tissue. The device of the present invention comprises a laser generator, an ultrasound generator, an ultrasound transducer, a handpiece containing a transducer tip at the distal end of the ultrasound transducer, and a laser tip. Ultrasonic and light radiation is directed into the tissue being treated. A cryogenic solution is circulated through the ultrasound tip to transfer thermal energy away from the tissue to preserve the tissue being treated by providing a synergistic effect with the ultrasonic radiation and light radiation.

### BACKGROUND OF THE INVENTION

The present invention relates to therapeutic and cosmetic treatment of a patient's skin and underlying adipose tissue and more particularly, to apparatuses and methods using combinations of ultrasound, lasers and cryogenic energy to rejuvenate and condition skin and related tissue.

Skin problems and disorders are common complaints afflicting individuals. Skin disorders frequently affect multiple layers of the skin and adjacent adipose tissue. Skin problems and complaints may be hereditary, caused by disease or due to a persons aging.

Research shows that there are, in fact, two distinct types of aging. Aging caused by the genes we inherit is called intrinsic aging. The other type of aging is known as extrinsic aging and is caused by environmental factors, such as exposure to the sun's rays. Intrinsic aging, also known as the natural aging process, is a continuous process that normally begins in the mid-20s for an individual. Within the skin, collagen production slows, and elastin, the substance that enables skin to snap back into place, has a bit less spring. Dead skin cells do not shed as quickly and turnover of new skin cells may decrease slightly. A number of extrinsic factors often act together with the normal aging process to prematurely age our skin. Most premature aging is caused by sun exposure. Other external factors that prematurely age our skin are repetitive facial expressions, gravity, sleeping positions, and smoking.

Although skin problems may be age related, other common skin disorders and cosmetic complaints of a hereditary or disease related may include removal or treatment of hair, wrinkles, scars, warts, spider veins, adipose tissue, non-metastsis melanomas, basilomas, human papillomaviruses, and various pre-cancerous or cancerous skin growths. Typical methods for treating skin disorders include surgical removal, lasers, high intensity ultrasound, chemical peeling, cryogenic destruction of diseased tissue, and various electrical treatments or the skin and underlying adipose tissue.

The disclosed embodiment teaches a laser/ultrasound system that may be used with cryogenic energy to rejuvenate and treat skin problems.

### BRIEF SUMMARY OF THE INVENTION

The present invention is directed towards apparatuses and methods for the rejuvenation of skin and the treatment of selected disorders and complaints. The invention is particularly applicable to cosmetic procedures related to skin and underlying adipose tissue that affects the skin such as liposuction. Delivering light, ultrasonic and cryogenic energies simultaneously and/or sequentially, the present invention may be used to stimulate, modify or destroy tissue. Combining the delivery of light, ultrasonic and cryogenic energy during treatment, the present invention may provide advantages over existing methods and devices for removing unwanted and/or diseased tissue.

The light used for skin treatment may be in the ultraviolet spectrum below 400 nm, the visible spectrum of light, 400 nm (violet) to 700 nm (red) or in the infrared spectrum, above 700 nm.

The light may be from a laser source (collimated) or non-collimated broad wavelength from a high intensity source such as a zenon light. Wavelength and optical power are the most important properties when considering the interaction of light with tissue for medical applications.

When laser energy hits a target tissue, it may be transmitted, reflected, adsorbed or scattered. For there to be a biologic effect on a target tissue, the energy must be absorbed. Each tissue has specific absorption characteristics base on its composition and chromophore content. The principal chromophores present in skin tissue are hemoglobin, melanin, water, lipid and protein.

Infrared light is absorbed primarily by water, while visible and ultraviolet light are primarily absorbed by hemoglobin and melanin, respectively. As wavelength decreases toward the violet and ultraviolet, scatter or absorption from covalent bonds in protein limits penetration depth in this range. Ultrasound energy may be used to increase the penetration depth without increasing laser energy applied.

In order to target a specific tissue, one should select a wavelength which is strongly absorbed by a chromophore present in that tissue. Most medical laser applications depend on the absorption of laser light to heat the target tissue. To prevent undesirable thermal injury to adjacent tissue, light can be applied in suitably timed pulses related to the size of the target structure.

The thermal relaxation time of a given structure is the time needed for 50% of the heat generated by absorption of a laser pulse to diffuse into the surrounding tissue, and is approximately equal to the square of the diameter of the target structure. The thermal containment time is the pulse width in which all of the heat is confined to the target and is approximately 25% of the thermal relaxation time. Ultrasound energy can be used to influence the thermal relaxation time allowing variability in the laser operating parameters.

With proper selection of the wavelength, exposure time, and intensity of the incident laser energy, the biologic effect on the target tissue can be optimized and undesirable collateral effect on adjacent tissue can be minimized. With the inclusion of cryogenic cooling and ultrasound energy, the efficacy of the laser can be greatly enhanced.

Extremely short (nanosecond domain) pulses strongly absorbed by a chromophore can induce extremely rapid heating and formation of an expanding thermal plasma. As the plasma collapses, the shock wave causes mechanical disruption of the target. The dwell time on tissue is too short for significant thermal effects on adjacent tissue. This photomechanical effect is exploited by Q-Switched medical lasers for treatment of tattoos and certain pigmented lesions.

Most medical applications involve the selective absorption of light energy using a longer (micro to millisecond domain) pulse width to cause rapid but selective heating of the target tissue with thermal injury. The biologic effect of tissue coagulation or ablation is exploited for laser resurfacing, treatment of vascular lesions, and laser hair removal.

Laser energy can interact directly or indirectly with chemical structures within tissue. Noble gas-halide, or Excimer lasers for LASIK refractive surgery exploit ultraviolet laser energy's ability to disrupt covalent bonds non-thermally in corneal protein. In Photodynamic Therapy (PDT), laser or narrowband light energy can trigger a chemical reaction directly by interacting with endogenous photosensitizing compounds in cells.

Low level laser or narrowband light has been used with varying success to modulate cellular activity to achieve a biological effect such as stimulation of hair growth, collagen remodeling, accelerated wound healing, etc. In most cases the mechanism of action remains unclear, although changes in mitochondrial activity or cell membrane permeability may be responsible. Accepted medical applications include collagen remodeling for photoaged skin, anti-inflammatory treatments, and blue light therapy for acne treatments.

The quantity of energy that can be applied to the target must be sufficient to achieve the desired effect, but not enough to cause collateral damage on adjacent tissues. Assuming proper selection of wavelength and pulse width, absorption from competing chromophores, scattering of light in tissue, and surface reflection must be considered.

Absorption from competing chromophores can be managed by cooling the structure containing the competing chromophore to minimize collateral thermal injury. A common clinical situation is protecting melanin-bearing epidermis while targeting melanin-bearing hair follicles during laser hair removal. The shorter epidermal thermal relaxation time allows heat to diffuse more quickly, and the lower initial temperature increases the threshold of epidermal injury, allowing higher energies to be safely applied to the hair follicle.

Light scattering broadens the incident beam. Increasing the spot size keeps scattered photons in the beam path to the target area, increasing the energy density in the target volume and making them available to engage the desired target structures. Doubling the spot diameter increases the treatment volume eight times, so a lower applied energy can be used to achieve an effective energy density at the target.

By selecting the appropriate wavelength and pulse width, and properly delivering the applied energy, one can achieve a selective effect on target tissue.

The heat dissipated by the laser light produces destructive and/or regenerative changes within the chromophore containing tissue. This is known as selective photothermolysis. Selective photothermolysis means a tissue is specifically targeted with laser light energy without affecting the surrounding structures.

With the present invention, the light therapy is influenced by the application of ultrasound energy with the light energy. The laser/ultrasound system includes a laser portion and an ultrasound portion. The laser portion includes a laser generator to produce the light energy, a light wave guide to carry the light waves and a laser tip to release the light waves to the treatment area. The ultrasound portion comprises an ultrasound generator driving an ultrasound transducer. An ultrasound horn is mechanically coupled to the ultrasound transducer. The ultrasound horn consists of a shaft and an ultrasound tip. The ultrasound horn receives the ultrasound waves from the ultrasound generator and transmits the ultrasound waves to the distal end of the ultrasound tip.

For ease of use, the laser portion and the ultrasound portion are integrated to the extent possible. For example preferably a single handpiece would carry the laser tip and ultrasound tip. In fact, the wave guide of the laser portion may be internal to the ultrasound transducer. In an alternative embodiment, a cryogenic fluid may be used to spray the treatment site or to cool the laser and/or ultrasound tip.

The ultrasound tip and laser tip may or may not contact the patients skin. In another embodiment, a shield such as a ruby crystal or silicon glass may be used to separate the respective tips from the patient's skin. A lubricant or gel such as silicone based materials may be used to displace air between the ultrasound tip to modify the transmission characteristics to the patient's skin.

Ultrasound energy may be optimized to achieve the desired effects by effectively utilizing its various properties including; thermal treatment, cavitation, microstreaming and harmonic resonance. At higher intensities, the use of the thermal energy produced from the ultrasound waves and the focused cavitation and microstreaming effects are particularly effective at disrupting or destroying unwanted tissue.

It is an object of this invention to enhance the therapeutic effects of laser treatment of skin tissue with the therapeutic effects of ultrasound treatment.

It is an object of this invention to enhance the therapeutic effects of laser treatment of skin tissue with the therapeutic effects of ultrasound treatment for epidermal treatments.

It is an object of this invention to enhance the therapeutic effects of laser treatment of skin tissue with the therapeutic effects of ultrasound treatment for dermal treatments beneath the epidermis.

It is an object of this invention to enhance the therapeutic effects of laser treatment of tissue with the therapeutic effects of ultrasound treatment for adipose tissue beneath the dermis.

It is an object of this invention to provide a laser/ultrasound system that can include cryogenic therapy.

It is an object of this invention to provide a laser/ultrasound system that can enhance light penetration into tissue by ultrasound vibrations of the tissue.

It is an object of this invention to provide a laser/ultrasound system that can enhance laser therapy through the therapeutic pain relief of ultrasound therapy.

It is an object of this invention to provide a pulsed laser and pulsed ultrasound system having the energy pulsed timed for simultaneous emissions.

It is an object of this invention to provide a pulsed laser and pulsed ultrasound system having the energy pulsed timed for alternating emissions.

It is an object of this invention to provide a pulsed laser and pulsed ultrasound system having a cryogenic spray alternating with the ultrasound energy and/or laser energy pulses.

It is an object of this invention to provide a pulsed laser and pulsed ultrasound system having a cryogenic spray simultaneously pulsed with the ultrasound energy and/or laser energy pulses.

### BRIEF DESCRIPTION OF THE SEVERAL VIEWS OF THE DRAWING

FIG. 1 depicts a sectional view of the skin and adjacent underlying tissue.

FIG. 2 depicts a sectional view of the invention in relation to treating epidermal tissue.

FIG. 3 is a perspective view of one embodiment of the laser/ultrasound system.

FIG. 4 depicts an embodiment of the ultrasound tip and laser tip with a cryogenic spray.

FIG. 5 depicts an embodiment of the ultrasound tip and laser tip with simultaneous application of light and ultrasound waves.

FIG. 6 depicts an embodiment of the ultrasound tip and laser tip with a laser tip internal to an ultrasound tip and a lens disposed between the ultrasound tip and the tissue.

FIG. 7 depicts an embodiment of the ultrasound tip and laser tip with cryogenic cooling of the ultrasound tip.

FIG. 8 depicts an embodiment of the ultrasound tip and laser tip with the ultrasound tip internal to the laser tip.

### DETAILED DESCRIPTION OF THE INVENTION

The present invention relates to a laser/ultrasound system to provide rejuvenation and therapeutic effects on skin and underlying tissues.

The use of laser treatment systems is well known for the treatment of skin. A wide variety of lasers are currently available, the efficacy of which may be enhanced with the co-application of ultrasound energy. Several well known lasers include:

CO2 laser light is absorbed by water in the skin, hence, used for skin resurfacing, removal of benign skin tumors like warts, xanthelasma, mucous cysts, cherry angiomas, leukoplakia and for surgical cutting.

Nd:YAG lasers have an active medium of Neodymium in yttrium-aluminum-garnet and the wavelength is 1064 nm. NdYAG lasers have slight absorption in melanin and hemoglobin and are used for laser hair removal, laser vein treatments, laser photo rejuvenation, laser acne treatments and in laser skin surgeries.

Q Switched NdYAG lasers have strong absorption in dark tattoo inks, hence used in laser tattoo removal.

Er:YAG lasers have a wavelength of 2940 nm and the active medium is Erbium in yttrium-aluminum-garnet. It is absorbed by water in the skin and is used for skin resurfacing, laser photo-rejuvenation and for removal of skin growths.

Ruby lasers have a wavelength of 694 nm and contain chromium ions in aluminum oxide as the medium. Ruby laser light has very strong absorption in melanin and black and dark blue ink pigments. These are especially useful in tattoo removal. Laser hair removal and removal of pigmented (dark) skin lesions.

KTP or Potassium Titanyl Phosphate laser with 532 nm wavelength is a frequency doubled NdYAG laser with absorption by hemoglobin and melanin and used to remove vascular and pigmented skin lesions.

Alexandrite lasers operate at 755 nm, Q switched mode laser, used to remove blue, black and green tattoos and epidermal and dermal pigmentations as in melasma.

Diode lasers operate at different wavelengths. The absorbing chromophores are melanin and hemoglobin in the skin. Diode lasers are used for laser hair removal, dilated vein treatments, and laser photo-rejuvenation.

Dye lasers contain organic compounds in solution (often rhodamine) as the active medium and have wavelength activity between 400 to 800 nm. The target chromophores are hemoglobin and melanin pigment. Dye lasers are useful in treating vascular lesions and for non-ablative skin rejuvenation.

Excimer laser containing compounds of xenon, krypton and argon target proteins and water and have wavelengths between 190-350 nm. Excimer lasers are useful in the treatment of psoriasis and vitiligo.

Fractional lasers produce microscopic treatment zones and target specific depths in the dermis. These are especially useful for the treatment of acne scars, wrinkles, sun damaged skin, melasma etc. Wavelength is in the range of 1550 nm, and the target chromophore is water within the tissue.

Broadband or Intense pulsed light (IPL) devices are often used for hair removal, these are broadband light sources, therefore not lasers, use a cutoff filter to limit the spectrum to wavelengths longer than 640 nm, minimizing absorption by chromophores other than melanin. IPL devices typically are typically less expensive to manufacture than monochromatic lasers. These devices can be provided with ultrasound enhancements as described with respect to laser systems.

Medical lasers use the principle of selective photothermolysis to deliver the right amount of energy to the target tissue in a manner that spares injury to adjacent structures. Some medical lasers are application-specific; others are versatile devices suitable for a variety of applications. All are designed to deliver the correct wavelength at the right energy and pulse width for the task at hand. No one laser or light based device is suitable for all medical indications. All medical lasers offer a way to adjust the energy output and pulse width, and some are capable of multiple modes, for example “long” pulse (millisecond) or Q-switched (nanosecond) operation.

A delivery device or light wave guide gets the laser energy to the target. Typically, laser energy is delivered through a fiberoptic cable or articulating arm through an output device, such as a handpiece.

Because most medical laser applications involve the addition of heat to a target tissue, many medical lasers incorporate some sort of cooling device to protect the epidermis, and/or minimize patient discomfort. Cooling can applied before, during or after application of laser energy. Cryogen cooling using R-134a refrigerant delivered through a device incorporated in the laser handpiece, applied before, during or after the laser pulse or may be used to chill a shield or lens such as a sapphire window.

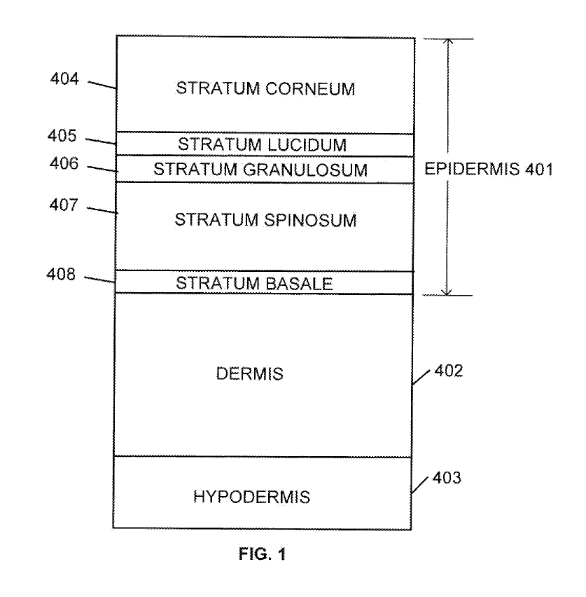

With regard to FIG. 1, a block diagram of skin layers. Skin consists of epidermis 401, dermis 402 and hypodermis 403. Epidermis 401 forms the waterproof, protective wrap over the body's surface and is made up of stratified squamous epithelium with an underlying basal lamina. Epidermis 401 is divided into several layers where cells are formed through mitosis at the innermost layers. These layers include; stratum corneum 404, stratum lucidum 405, stratum granulosum 406, stratum spinosum 407 and stratum basale 408.

The epidermis 401 contains no blood vessels, and cells in the deepest layers are nourished by diffusion from blood capillaries extending to the upper layers of the dermis 402. The main type of cells which make up the epidermis are Merkel cells, keratinocytes, with melanocytes and Langerhans cells also present. The daughter cells move up the strata changing shape and composition as they die due to isolation from their blood source. The cytoplasm is released and the protein keratin is inserted. They eventually reach the corneum and slough off. This process is called keratinization and takes place within about 27 days. This keratinized layer of skin is responsible for keeping water in the body and keeping other harmful chemicals and pathogens out, making skin a natural barrier to infection. The epidermis contains no blood vessels, and is nourished by diffusion from the dermis 402.

The dermis 402 is the layer of skin beneath the epidermis that consists of connective tissue and cushions the body from stress and strain. The dermis 402 is tightly connected to the epidermis by a basement membrane. It also harbors many mechanoreceptor/nerve endings that provide the sense of touch and heat. It contains the hair follicles, sweat glands, sebaceous glands, apocrine glands, lymphatic vessels and blood vessels. The blood vessels in the dermis provide nourishment and waste removal from its own cells as well as from the stratum basale of the epidermis.

The dermis 402 is structurally divided into two areas: a superficial area adjacent to the epidermis, called the papillary region, and a deep thicker area known as the reticular region.

The papillary region is composed of loose areolar connective tissue. It is named for its fingerlike projections called papillae, that extend toward the epidermis. The papillae provide the dermis with a “bumpy” surface that interdigitates with the epidermis, strengthening the connection between the two layers of skin.

The reticular region lies deep in the papillary region and is usually much thicker. It is composed of dense irregular connective tissue, and receives its name from the dense concentration of collagenous, elastic, and reticular fibers that weave throughout it. These protein fibers give the dermis its properties of strength, extensibility, and elasticity. Also located within the reticular region are the roots of the hair, sebaceous glands, sweat glands, receptors, nails, and blood vessels.

The hypodermis 403 is not part of the skin, and lies below the dermis. Its purpose is to attach the skin to underlying bone and muscle as well as supplying it with blood vessels and nerves. It consists of loose connective tissue and elastin. The main cell types are fibroblasts, macrophages and adipose tissue consisting largely of lipids.

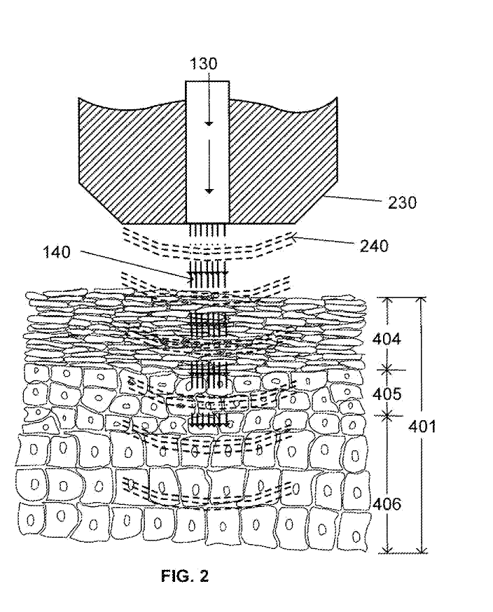

FIG. 2 depicts a sectional view of the invention in relation to treating epidermal tissue. Ultrasound waves 240 from the ultrasound tip 230 pass into the epidermis 401. The ultrasound energy vibrates the various layers of the epidermis 401 modifying the intercellular distances and relationships. Disrupting the cellular structure such as the tightly packed cells of the stratum corneum 404 allow the light waves 140 to pass further into the epidermis 401 at a given laser dosage.

Furthermore, the vibration of the ultrasound energy modifies the heat transfer properties of the light absorbing cellular components in relation to dissipating heat to surrounding tissue. For example in hair removal treatments it is often preferred to dissipate the heat adsorbed by the hair into the surrounding follicle to achieve permanent hair removal by inactivating the follicle rather than just temporary hair removal achieved if the follicle remains viable and only the hair cells are destroyed. Alternatively in some treatments it is desirable to have a more uniform temperature distribution throughout the tissue to avoid inactivating any cells even though the energy is being adsorbed by a particular chromophore.

A number of laser treatment therapies are painful requiring general and/or local anesthesia. The analgesic effects of ultrasound through the effect of ultrasound interaction with nerve cells make use of an important feature of ultrasound to counteract a side effect of some laser therapy making ultrasound an important complement to laser therapy.

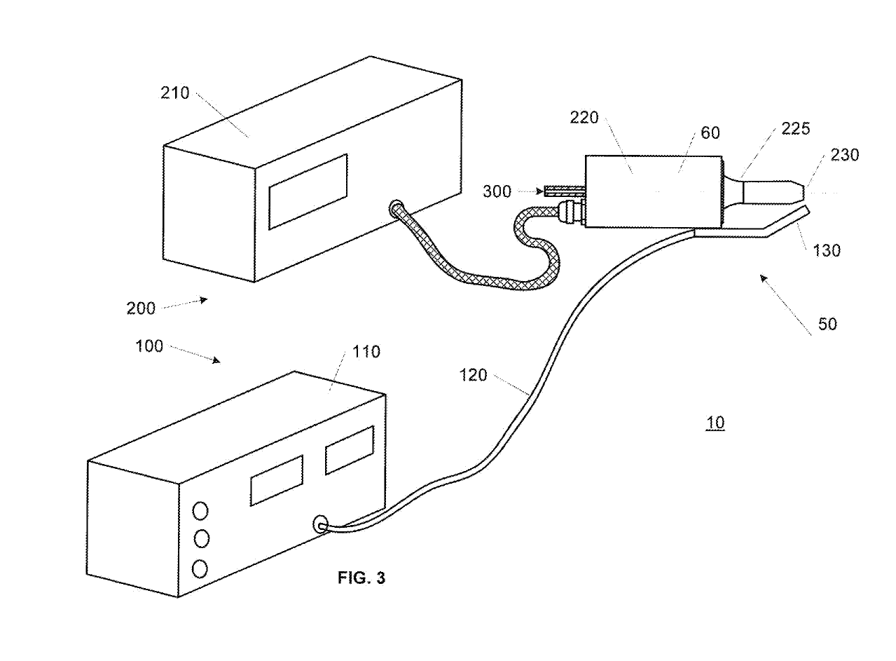

With respect to FIG. 3, a general view of an embodiment of the apparatus is shown. Although shown separately in FIG. 3, the ultrasound portion 100 and the laser portion 100 are generally combined physically in the same cabinet to appear to be as a single unit. The apparatus of the present invention may include a handpiece 50 with a housing 60 surrounding an ultrasound transducer 220 as shown in FIG. 3. The housing 60 provides a surface for the surgeon to hold for manipulation of the device over the skin. The housing 60 also may provide dampening and isolation so that the heat, electrical and mechanical energy emitted from the ultrasound transducer 220 or the laser tip 130 do not interfere with the operator's control of the device. The housing 60 may extend over a portion of the ultrasound transducer tip 230 to insulate the ultrasound transducer tip 230 and isolate portions of the proximal end of the ultrasound transducer tip 230 and/or laser tip 130 from contact with tissue 400.

The laser portion 100 includes a laser generator 110 providing the desired light to a light wave guide 120 which may be a fiber optic cable. The light wave guide 120 terminates at a laser tip 130 from which light waves are directed to the skin tissue. The light waves may be continuous waved (cw) or may be pulsed using a pulsed laser generator 110 which generates and emits light in pulses. A cw generator may also produce a pulsed light by simply mechanically shuttering the emitted light to intermittently block the light beam.

The ultrasound transducer 220 is driven by an ultrasound generator 210. The ultrasound generator 210 and laser generator 110 are typically powered with standard AC current which is electrically connected to an ultrasound transducer 220 through a cable and activated with a hand or foot operated switch. Ultrasound transducer 220 may be driven with a continuous wave or pulsed frequency signal supplied by ultrasound generator 210. Driving transducer 220 with a continuous wave tends to induce the release of standing waves from the various surfaces of tip 230, while a pulsed frequency reduces or avoids the release of standing waves. The pulsed frequency signal generates less heat, cavitation and streaming currents, and may increase the longitudinal force of the induced vibrations as a result of the on/off cycle changes. The electrical signal may be changed depending on the desired features of the released ultrasound waves for the particular application. The ultrasonic transducer 220 is pulsed according to a driving signal generated by the ultrasound generator 210 and transmitted to the ultrasonic transducer 220 by cable. The driving signals, as a function of time, may be rectangular, trapezoidal, sinusoidal, triangular or other signal types as would be recognized by those skilled in the art.

The ultrasound generator 210 may also be programmable to provide a rapid pulsed on-off signal to the ultrasound transducer 220 to modify the vibrational interaction between the transducer tip 230 and the tissue which may control and limit friction, tissue attachment, standing wave production and temperature rise within the tissue. This pulsed signal may vary between 0 to 100% depending on the application.

The distal end of the ultrasound transducer 220 is attached to an ultrasound horn 225 for conditioning and directing the ultrasonic energy through an transducer tip 230 to the tissue area selected for treatment. The ultrasound waves are emitted at a frequency and amplitude. The ultrasonic frequency may be used in embodiments that include low frequency or high frequency embodiments that operate within the range of 15 kHz and 20 mHz. The preferred frequency range for the transducer tip 230 is 15 kHz to 50 kHz with a recommenced frequency of approximately 30 kHz.

The amplitude of the ultrasonic waves may be between 1 micron and 250 microns with a preferred amplitude in the range of 10 to 50 microns and a recommended amplitude of 20 microns.

The ultrasound energy selected to penetrate to the subcutaneous tissue layer of the skin is generally applied for a duration of time from about 1 millisecond to about 30 minutes, such that the ultrasonic radiation damages the subcutaneous tissue, and allows for the natural re-growth of new cellular structure. In this manner, the ultrasound waves may be used to supplement or modify the effect of the laser treatment.

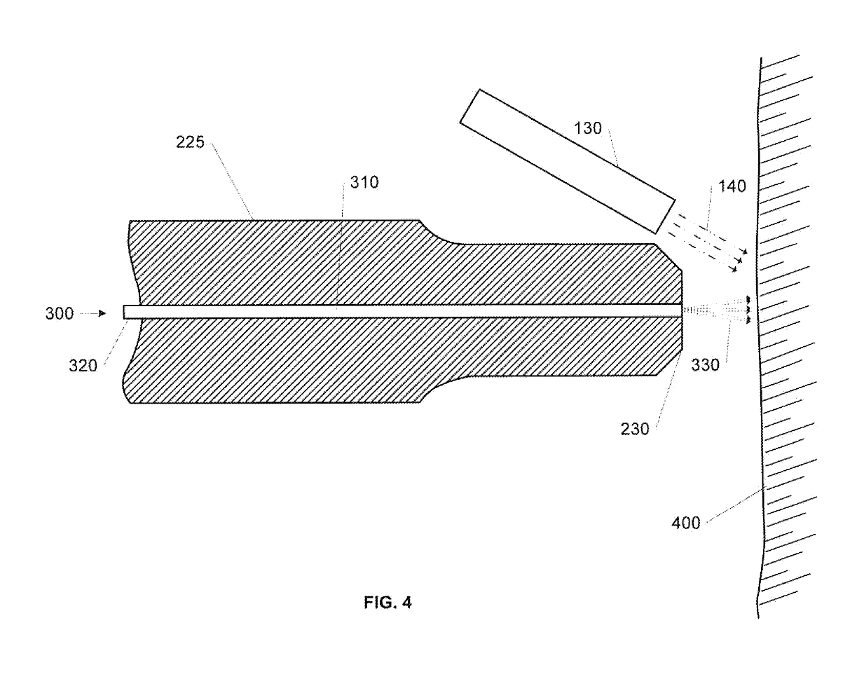

FIG. 4 depicts an embodiment of the distal end of the ultrasound tip 230 and laser tip 130 with a cryogenic spray 330. With this embodiment, an internal tube is used within the ultrasound horn 225 to transport cryogenic fluid 310 from a cryogenic source 300. The cryogenic fluid 310 may be emitted from the ultrasound tip as a cryogenic spray 330 on the tissue 400 or skin surface. The cryogenic fluid 310 is also used to remove heat generated from the ultrasound energy within the transducer tip 230. The cryogenic spray may be continuous or pulsed. If pulsed, the pulses may be timed to occur simultaneously with an ultrasound or laser pulse, or sequentially between pulses. To avoid dispersion and diffraction of the laser waves 140, generally the cryogenic spray 330 would not be simultaneous with the light emissions. In FIG. 4 the cryogenic spray 330 and light wave 140 are shown as being simultaneous. The dispersion or diffraction of light may not be an issue particularly when the laser light 140 is focused toward underlying or internal tissue 400 while the cryogenic spray will not pass beyond the skin surface.

A cryogenic source 300 may be used to supply the cryogenic fluid 310. One or more delivery tubes are typically used to deliver the cryogenic fluid 310 from the cryogenic source to the transducer tip 230. The cryogenic source 300 may include a refrigeration system that recycles the cryogenic fluid 310 through the transducer tip 230 or it may be a vented once-through system such as those using liquid nitrogen or liquid carbon dioxide for example.

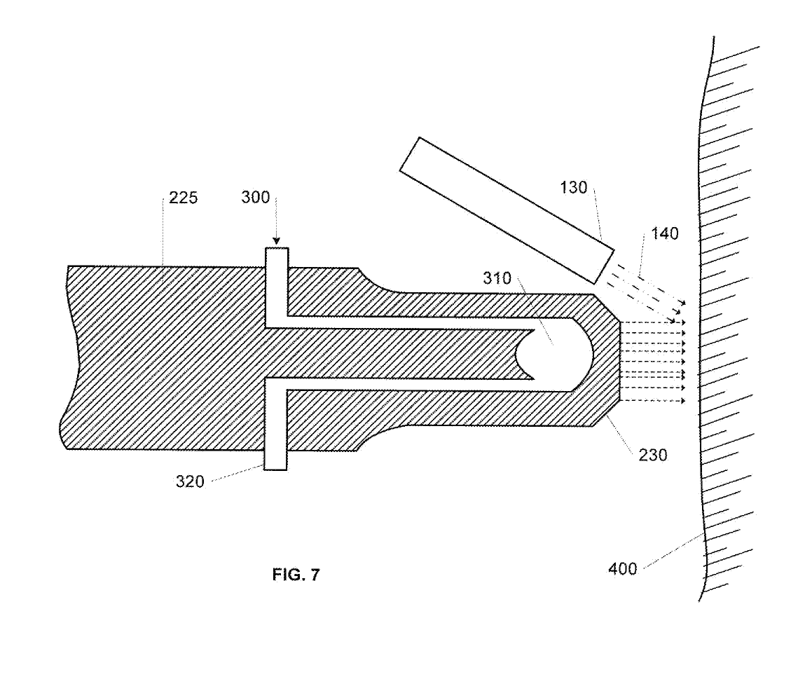

A cryogenic source 300 may be a refrigeration system capable of recycling the cryogenic fluid 310 through the transducer tip 230. The interior passage layout of FIG. 7 may be preferred for use with a refrigeration system. Typical examples include liquid/vapor compression type units that utilize a condensation-evaporation cycle or Joule-Thompson type refrigeration systems. Joule-Thompson refrigeration systems utilize a pressurized gas that cools when decompressed such as, but not limited to, argon, air, carbon tetra-fluoride, xenon, krypton, nitrous oxide or carbon dioxide. The gas used as a cryogenic fluid 310 is pressurized and then decompressed in an expansion chamber such as a chamber portion resulting in cooling of the gas within the transducer tip 230.

The ultrasound tip 230 may also contain one or more temperature sensors which may control the flow rate of cryogenic fluid 310 through the ultrasound transducer tip 230 to maintain a constant preselected temperature at the tip regardless of the ultrasound energy emitted from the tip. A temperature controller may also be used to vary the temperature through a manually controllable or a preprogrammed cycle. The ultrasound tip 230 may then be placed adjacent to the tissue 400 to be ablated to create an area of frozen tissue to the distal end of the ultrasound tip 230.

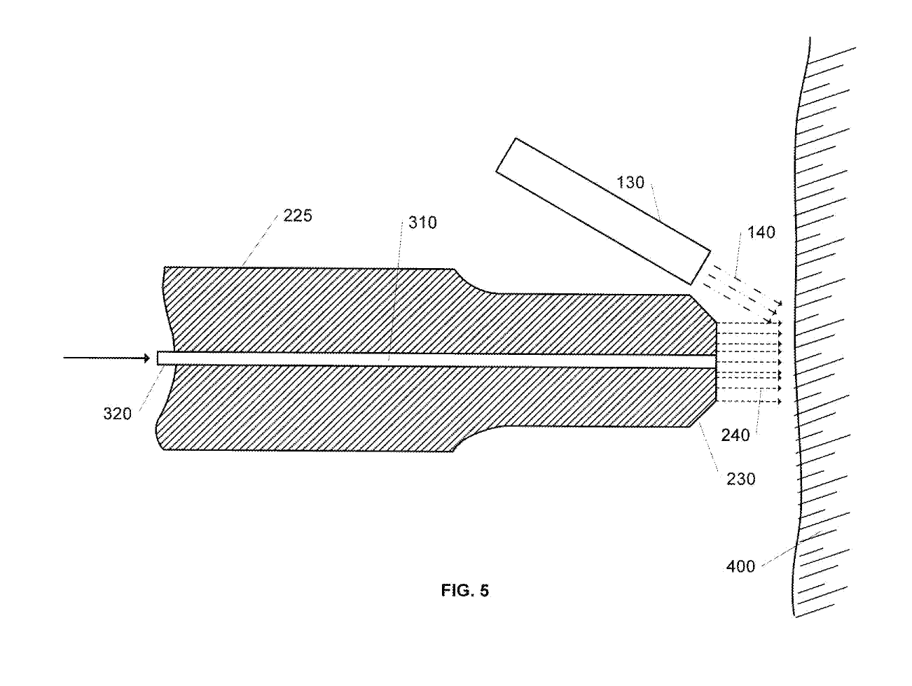

FIG. 5 depicts an embodiment of the ultrasound tip 230 and laser tip 130 with simultaneous application of light waves 140 and ultrasound waves 240.

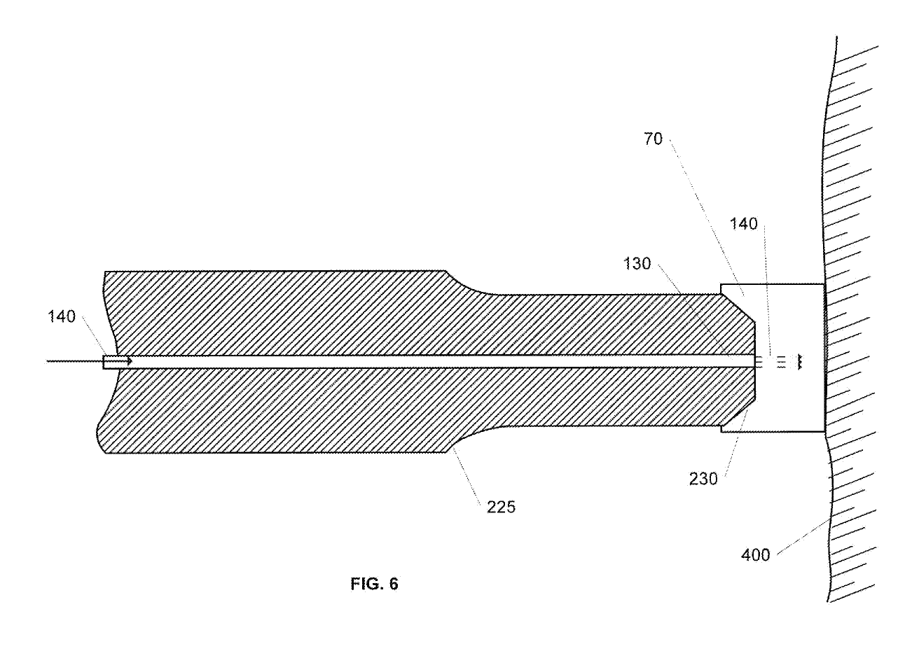

FIG. 6 depicts an embodiment of the ultrasound tip 230 and laser tip 130 with a laser tip 130 internal to an ultrasound tip 230. In this embodiment the light waves 140 pass internally within the ultrasound horn 225. A lens 70 or shield is shown between the tissue 400 and the ultrasound tip 230. The lens 70 would be compatible with transmitting the ultrasound and light waves. Typical materials of construction include quartz, sapphire or silicone materials.

FIG. 7 depicts an embodiment of the ultrasound tip 230 and laser tip 130 with cryogenic cooling of the ultrasound tip 230. The cryo-transfer tube 320 through which cryogenic fluid 310 flows through the ultrasound horn 225 may include an expansion shown as a chamber portion in FIG. 7. Although the ultrasound tip 230 and laser tip 130 are shown as separated from the tissue 400. In an alternative embodiment, the ultrasound tip 230 and/or laser tip 130 may be placed in direct contact with the tissue 400.

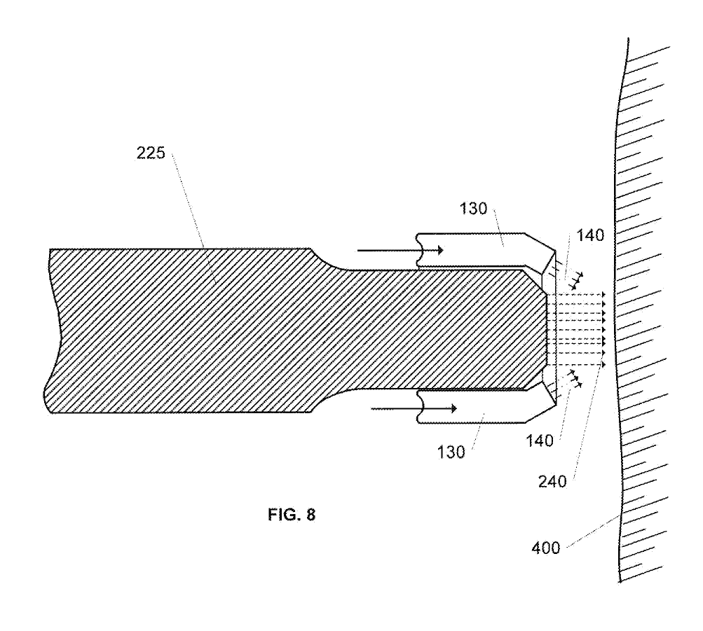

FIG. 8 depicts an embodiment of the ultrasound tip 230 and laser tip 130 with the ultrasound tip 230 internal to the laser tip 130. In this embodiment the ultrasound tip 230 and laser tip 130 may be in contact with the tissue 400, non-contact with the tissue 400 or alternatively in indirect contact with the tissue using a lens 70 or shield between the tissue and the ultrasound tip 230 and/or laser tip 130.

In ultrasound/laser assisted liposuction, a direct or indirect contact using a lens 70 embodiments allow very efficient cryogenic cooling of the skin. This allows the skin tissue to be directly cooled and therefore preserved from heat damage while lower adipose tissues are disrupted to allow easier removal through a cannula.

Although specific embodiments of apparatuses and methods for the treatment and rejuvenation of skin using an ultrasound assisted laser have been illustrated and described herein, it will be appreciated by those of ordinary skill in the art that any arrangement, combination, and/or sequence that is calculated to achieve the same purpose may be substituted for the specific embodiments shown. It is to be understood that the above description is intended to be illustrative and not restrictive. Combinations of the above embodiments and other embodiments as wells as combinations and sequences of the above methods and other methods of use will be apparent to individuals possessing skill in the art upon review of the present disclosure.

The scope of the claimed apparatus and methods should be determined with reference to the appended claims, along with the full scope of equivalents to which such claims are entitled.

I claim:

1. An ultrasound laser device comprising:
a laser generator producing light waves;
a light wave guide transporting the light wave to a laser tip;
an ultrasound generator driving;
an ultrasound transducer generating ultrasound waves;
an ultrasound tip receiving the ultrasound wave from the ultrasound transducer; and
a handpiece containing the laser tip and the ultrasound tip.

2. The device of claim 1 having a cryogenic spray.

3. The device of claim 1 having a housing at least partially enclosing the ultrasound transducer and the light wave guide.

4. The device of claim 1 wherein the ultrasound tip is in direct contact with a tissue.

5. The device of claim 1 wherein the ultrasound tip does not contact a tissue.

6. The device of claim 1 also having a lens disposed on the laser tip.

7. The device of claim 1 wherein the ultrasound tip is at least partially within the laser tip.

8. The device of claim 1 wherein the ultrasound generator provides a signal selected from the group consisting of sinusoidal, trapezoidal, triangular or rectangular.

9. The device of claim 1 wherein the ultrasound generator provides a pulsed signal.

10. The device of claim 1 wherein the ultrasound waves are emitted at a frequency ranging between 16 kHz and 20 mHz.

11. The device of claim 1 wherein the ultrasound waves are emitted at a wavelength between 1 micron and 250 microns.

12. The device of claim 1 wherein the laser tip is disposed within the ultrasound tip.

13. The device of claim 1 wherein the ultrasound waves are continuous.

14. The device of claim 1 wherein the light waves are continuous.

15. The device of claim 1 wherein the light waves are pulsed.

16. The device of claim 1 wherein the ultrasound waves are emitted sequential to the light waves.

17. The device of claim 1 wherein the ultrasound waves are emitted simultaneous with the light waves.

18. The device of claim 2 wherein the light waves are emitted sequential to the cryogenic spray.
